(19) United States
(12) Patent Application Publication
(10) Pub. No.: US 2020/0315869 A1
(43) Pub. Date: Oct. 8, 2020

(54) ABSORBER AND ABSORBENT ARTICLE

(71) Applicant: KAO CORPORATION, Tokyo (JP)

(72) Inventor: Ryota KURAMAE, Utsunomiya-shi, Tochigi (JP)

(73) Assignee: KAO CORPORATION, Tokyo (JP)

(21) Appl. No.: 16/954,685

(22) PCT Filed: Dec. 29, 2017
(86) PCT No.: PCT/JP2017/047402

§ 371 Date: Jun. 17, 2020

(51) Int. Cl. A61F 13/53; A61F 13/15

(52) U.S. Cl. CPC A61F 13/53; A61F 13/15577; A61F 2013/53975; A61F 2013/530635; A61F 2013/53062

(57) ABSTRACT

An absorbent member (1) according to the present invention includes: an absorbent sheet (2) that includes a substrate layer (20) and an absorbent layer (21) that includes water-absorbent polymers (P1) that are fixed to one side of the substrate layer (20); and an entangled fiber layer (3). The water-absorbent polymers (P1) of the absorbent layer (21) and the entangled fiber layer (3) are adjacent to each other. The water-absorbent polymers (P1) of the absorbent layer (21) are preferably fixed to one side of the substrate layer (20) using a hot melt adhesive. The absorbent member (1) configured as described above has excellent absorbency and shape stability while being thin.

### TECHNICAL FIELD

The present invention relates to an absorbent member that is used to absorb a liquid and is suitable as a constituent member that constitutes an absorbent article such as a sanitary napkin or a disposable diaper.

### BACKGROUND ART

An absorbent sheet in which water-absorbent polymers are fixed to one side of a substrate sheet is known as an absorbent member of an absorbent article. An absorbent sheet that has the above-described configuration is used mainly in a thin absorbent article because it is thin and is not bulky as compared with an absorbent member that includes a fiber stack made of a fiber material such as pulp fibers. For example, Patent Literatures 1 and 2 each disclose an absorbent sheet in which a plurality of water-absorbent polymer particles are fixed between two non-woven fabrics using a hot melt adhesive.

### CITATION LIST

### Patent Literature

Patent Literature 1: WO 2001/089439

Patent Literature 2: JP 2010-529879A

### SUMMARY OF INVENTION

The present invention provides an absorbent member including: an absorbent sheet that includes a substrate layer and an absorbent layer that includes water-absorbent polymers that are fixed to one side of the substrate layer; and an entangled fiber layer, wherein the water-absorbent polymers of the absorbent layer and the entangled fiber layer are adjacent to each other.

Also, the present invention provides an absorbent article that includes the above-described absorbent member of the present invention.

### BRIEF DESCRIPTION OF DRAWINGS

FIG. 1 is a perspective view schematically showing an absorbent member according to an embodiment of the present invention.

FIG. 2 shows a cross section taken along the line I-I shown in FIG. 1, and is a cross-sectional view schematically showing a cross section taken along the thickness direction of the absorbent member shown in FIG. 1.

FIGS. 3(a) and 3(b) are cross sectional views schematically showing cross sections of absorbent members according to other embodiments of the present invention taken along the thickness direction thereof (corresponding to FIG. 2).

FIG. 4 is a cross sectional view schematically showing a cross section of an absorbent member according to yet another embodiment of the present invention taken along the thickness direction thereof (corresponding to FIG. 2).

### DETAILED DESCRIPTION OF THE INVENTION

The absorbent sheets disclosed in Patent Literatures 1 and 2 are thinner than a fiber stack of pulp fibers conventionally used as an absorbent member used in an absorbent article, and thus they can satisfy the need for thin absorbent members and are useful for improving the wearing comfort, portability, and the like of the absorbent article. However, because the amount of pulp fibers, which are capable of absorbing a liquid, contained in the absorbent sheets is small, they tend to be inferior to a fiber stack in terms of absorbent capacity, absorption speed, and the like. Accordingly, there is room for improvement in terms of absorbency.

On the other hand, the fiber stack of pulp fibers is thicker than the absorbent sheet, and thus is superior to the absorbent sheets in terms of absorbent capacity, absorption speed, and the like. However, when the fiber stack of pulp fibers is used in an absorbent article, due to the thickness of the fiber stack of pulp fibers, the wearing comfort of the absorbent article may decrease. In addition, this type of fiber stack of pulp fibers has poor shape stability, particularly after absorbing a liquid. Accordingly, in the case where it is used in an absorbent article, for example, when an external force is applied to the fiber stack due to movement of the wearer after the fiber stack has absorbed a body fluid, the outer shape of the fiber stack may be deformed and break apart, which, as a result, may cause disadvantages such as discomfort for the wearer and a reduction in absorbency.

As described above, conventional absorbent members for absorbent articles have advantages and disadvantages. An absorbent member that can satisfy the need for thin absorbent members and has excellent absorbency and shape stability after absorbing a body fluid has not yet been provided.

Accordingly, it is an object of the present invention to provide an absorbent member that is thin and has excellent absorbency and shape stability, and an absorbent article that includes the absorbent member.

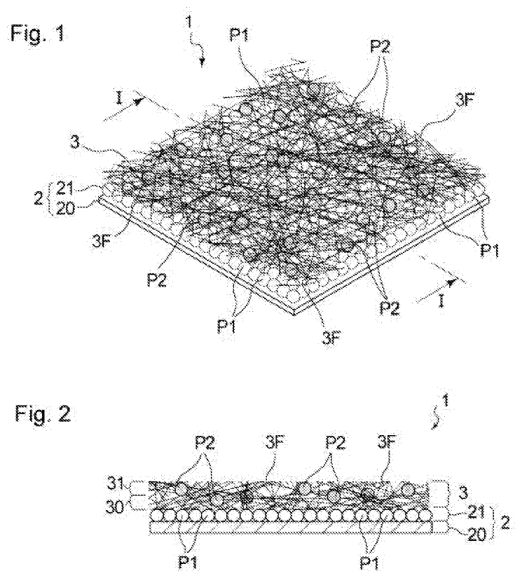

Hereinafter, the present invention will be described by way of a preferred embodiment of the present invention with reference to the drawings. FIGS. 1 and 2 show an absorbent member 1 that is an absorbent member according to an embodiment of the present invention. The absorbent member 1 includes an absorbent sheet 2 and an entangled fiber layer 3. More specifically, the absorbent member 1 has a stack structure including an absorbent sheet 2 and an entangled fiber layer 3. The absorbent sheet 2 includes a substrate layer 20 and an absorbent layer 21 that contains water-absorbent polymers P1 that are fixed to one side of the substrate layer 20. The entangled fiber layer 3 is a fiber assembly in which a plurality of fibers 3F are entangled with each other without being fused to each other.

A main feature of the absorbent member 1 according to the present embodiment is that, as shown in FIGS. 1 and 2, the water-absorbent polymers P1 of the absorbent layer 21 in the absorbent sheet 2 and the entangled fiber layer 3 are adjacent to each other. That is, in the absorbent member 1, the absorbent layer 21 and the entangled fiber layer 3 directly and completely overlap each other without any member being interposed therebetween, and the water-absorbent polymers P1 of the absorbent layer 21 are in contact with the constituent fibers 3F of the entangled fiber layer 3.

Assuming that an absorbent member composed only of an entangled fiber layer 3 is used in an absorbent article such as a disposable diaper, when an external force is applied to the entangled fiber layer 3 after the absorbent member, or in other words, the entangled fiber layer 3, has absorbed a body fluid such as urine due to movement of the wearer of the absorbent article, the outer shape of the entangled fiber layer 3 may be deformed and break apart, which, as a result, may cause disadvantages such as discomfort for the wearer and a reduction in absorbency. In contrast, the absorbent member 1 according to the present embodiment includes, in addition to the entangled fiber layer 3, the absorbent sheet 2 that is stacked on the entangled fiber layer 3. Furthermore, because the entangled fiber layer 3 is adjacent to the water-absorbent polymers P1 of the absorbent layer 21 in the absorbent sheet 2, even if an external force is applied to the entangled fiber layer 3 after absorbing a liquid and the entangled fiber layer 3 is about to break apart, the water-absorbent polymers P1 that are fixed to the substrate layer 20 and are in contact with the entangled fiber layer 3 absorb the liquid, and turn into gel and become adhesive, and due to the adhesiveness, the water-absorbent polymers P1 bond to the entangled fiber layer 3 (constituent fibers 3F). Accordingly, the shape stability of the entangled fiber layer 3 is enhanced, the entangled fiber layer 3 is unlikely to break apart, and the above-described disadvantages are eliminated.

As described above, as long as at least the water-absorbent polymers P1 of the absorbent layer 21 and the entangled fiber layer 3 are provided adjacent to each other, due to the adhesiveness of the water-absorbent polymers P1 after absorbing a liquid, the shape stability of the entangled fiber layer 3 can be enhanced, and a situation can be suppressed in which the entangled fiber layer 3 breaks apart. That is, in order to avoid a situation in which the entangled fiber layer 3 that has absorbed a liquid breaks apart, it is sufficient that the water-absorbent polymers P1 and the entangled fiber layer 3 (constituent fibers 3F) are in contact with each other. The water-absorbent polymers P1 and the entangled fiber layer 3 do not necessarily have to be bonded to each other with an adhesive. In the case where the water-absorbent polymers P1 and the entangled fiber layer 3 are bonded to each other with an adhesive, further improvement in the shape stability of the entangled fiber layer 3 can be expected. In this case, care is preferably taken to select the type of adhesive, the position of the adhesive, and the like, such that the adhesive does not impair the liquid absorbency of the absorbent member 1.

In the present embodiment, the surfaces of the substrate layer 20 are substantially (macroscopically) flat. The water-absorbent polymers P1 are fixed to one of the flat surfaces of the substrate layer 20, and the water-absorbent polymers P1 are not fixed to the other surface of the substrate layer 20.

In the present embodiment, the water-absorbent polymers P1 are in the form of particles, and a plurality of particle-shaped water-absorbent polymers P1 are fixed to one side of the substrate layer 20. There is no particular limitation on the shape of the particle-shaped water-absorbent polymers P1. The shape of the particle-shaped water-absorbent polymers P1 may be, for example, spherical, aggregate, bale-like, or amorphous. There is no particular limitation on the average particle size of the particle-shaped water-absorbent polymers P1, but from the viewpoint of balancing fixation and absorbency, the average particle size of the particle-shaped water-absorbent polymers P1 is preferably 50 μm or more, more preferably 200 μm or more, and preferably 800 μm or less, and more preferably 500 μm or less. In the present invention, there is no particular limitation on the form of the water-absorbent polymers P1. The water-absorbent polymers P1 may be in the form of, other than particles, for example, fibers, a sheet, or the like. The water-absorbent polymers P1 may be in the form of sheet shreds obtained by cutting the water-absorbent polymers in the form of a large sheet that has a relatively large area into small pieces that have a relatively small area such as strips.

As the water-absorbent polymers P1, any water-absorbent polymers that can be used in this type of absorbent sheet can be used without any particular limitation. Examples include polyacrylic acid soda, an acrylic acid-vinyl alcohol copolymer, cross-linked polyacrylic acid soda, a starch-acrylic acid graft polymer, an isobutylene-maleic anhydride copolymer, saponified products thereof, potassium polyacrylate, cesium polyacrylate, and the like. These may be used alone or in a combination of two or more.

From the viewpoint of further improving the above-described effect of keeping the entangled fiber layer 3 from breaking apart provided by the water-absorbent polymers P1, the water-absorbent polymers P1 of the absorbent layer 21 are preferably highly adhesive when the water-absorbent polymers P1 have absorbed a liquid. Specifically, the water-absorbent polymers P1 have an adhesion rate of preferably, 0.5 or more, and particularly preferably 0.7 or more, the adhesion rate being measured using a method described below. Also, the upper limit for the adhesion rate of the water-absorbent polymers P1 measured using a method described below is preferably 0.95 or less, and more preferably 0.85 or less, from the viewpoint of suppressing a reduction in absorbency caused by gel blocking between water-absorbent polymers P1.

### Method for Measuring Adhesion Rate

1 g of a physiological saline solution is dropped onto a center portion of a piece of dry filter paper to wet the center portion, and 0.1 g of water-absorbent polymers acting as a measurement target are uniformly sprayed onto the center portion of the filter paper placed on a horizontal table. For example, the water-absorbent polymers can be uniformly sprayed onto a center portion (φ 100) of a piece of filter paper No. 4 A (φ 150) available from Advantec Toyo K.K after wetting the center portion using a physiological saline solution. One minute after spraying the water-absorbent polymers, the filter paper is supported at a position 5 mm above the table, with the filter paper being vertically inclined, and the support is removed to drop the filter paper. The operation of dropping the filter paper is repeated 100 times. From the total amount a of water-absorbent polymers detached from the filter paper during the dropping operations and the total amount b of water-absorbent polymers remaining on the filter paper, the adhesion rate of the water-absorbent polymers is calculated using the following equation.

Adhesion rate=b/(a+b)

In the adhesion rate measurement described above, in the case where the water-absorbent polymers used as a measurement target are in an unused state (in the case where the water-absorbent polymers are not fixed to a substrate layer and thus do not function as a constituent element of an absorbent sheet), the unused water-absorbent polymers are used as a measurement target. On the other hand, in the case where the water-absorbent polymers used as a measurement target function as a constituent element of an absorbent sheet, the water-absorbent polymers are collected from the absorbent sheet, and the collected water-absorbent polymers are used as a measurement target. That is, it is sufficient that the adhesion rate of the water-absorbent polymers P1 is within the above-described preferred range when the water-absorbent polymers P1 are in at least one of an unused state and a used state (a state in which the water-absorbent polymers P1 are fixed to the substrate layer 20).

The water-absorbent polymers P1 that have an adhesion rate of 0.5 or more can be obtained by, for example, in a known method for producing polyacrylic acid-based water-absorbent polymers such as a reverse-phase suspension polymerization method or an aqueous solution polymerization method, changing conditions such as reducing the amount of cross-linking agent added, or reducing the reaction time, so as to reduce the degree of cross-linking in the resulting water-absorbent polymers.

The fixation rate of the water-absorbent polymers P1 of the absorbent layer 21 to the substrate layer 20 is preferably 10% or more, and particularly preferably 30% or more. The fixation rate of the water-absorbent polymers P1 indicates the proportion of water-absorbent polymers P1 that do not detach from the absorbent sheet 2 even when the absorbent sheet 2 is placed in a physiological saline solution (0.9 mass % saline solution) stirred at a predetermined rotation speed, and may serve as an index for fixation of the water-absorbent polymers P1 that have absorbed a liquid and swollen to the substrate layer 20. The higher the value of the fixation rate, the stronger the fixation of the water-absorbent polymers P1 to the substrate layer 20 in the absorbent sheet 2, which indicates that the water-absorbent polymers P1 are unlikely to detach not only before use but also after absorbing a liquid. When the fixation rate of the water-absorbent polymers P1 to the substrate layer 20 is 10% or more, a large number of water-absorbent polymers P1 that is substantially the same as the number of water-absorbent polymers P1 before absorbing a liquid are fixed to the substrate layer 20 even after the water-absorbent polymers P1 have absorbed the liquid, and thus the above-described effect of keeping the entangled fiber layer 3 from breaking apart provided by the water-absorbent polymers P1 can be further improved. Also, the upper limit for the fixation rate of the water-absorbent polymers P1 to the substrate layer 20 in the absorbent layer 21 is preferably 90% or less, and more preferably 80% or less from the viewpoint of not impairing the flexibility of the absorbent sheet 2 after absorbing a liquid. When the fixation rate is too high, water-absorbent polymers P1 that have absorbed a liquid and swollen densely abut against each other, which may increase the rigidity of the absorbent sheet 2 and reduce the flexibility of the absorbent sheet 2. The fixation rate of the water-absorbent polymers P1 can be measured through the following steps 1 to 5.

Step 1: an absorbent sheet that has a 5 cm×5 cm square shape as viewed in a plan view is prepared as a measurement sample. The measurement sample is vertically suspended with an end portion thereof being held, and the weight (initial sample weight) of the measurement sample is measured.

Step 2: the measurement sample is fully immersed in a physiological saline solution, and the measurement sample is taken out from the physiological saline solution 30 minutes after immersion.

Step 3: a cylindrical stirring bar with a diameter of 35 mm and an axial length of 12 mm and 300 ml of physiological saline solution are placed in a 300 ml beaker, and the stirring bar is rotated at a rotation speed of 600±5 rpm using a magnetic stirrer to stir the physiological saline solution. The measurement sample obtained in step 2 is placed in the physiological saline solution while it is being stirred, and the measurement sample is taken out from the physiological saline solution 30 seconds after placing the measurement sample in the physiological saline solution.

Step 4: the wet measurement sample that was wetted in step 3 is placed in a constant temperature chamber with its inner temperature being set to 105° C., and left in the chamber for 12 hours, and thereafter the weight (sample weight after stirring) of the dried measurement sample is measured.

Step 5: initial water-absorbent polymer weight (W0) and water-absorbent polymer weight (W1) after stirring are calculated by subtracting the total weight of the members other than the water-absorbent polymers from the initial sample weight and the sample weight after stirring, respectively. Then, the water-absorbent polymer fixation rate of the measurement sample (absorbent sheet) is calculated using the following equation.

Fixation rate of water-absorbent polymers (%)=(W1/W0)×100

The reason that the measurement sample (an absorbent sheet that has a 5 cm×5 cm square shape as viewed in a plan view) is vertically suspended with an end portion thereof being held in step 1 is to remove non-fixed substances such as water-absorbent polymers that are provided on the substrate sheet without being fixed (for example, water-absorbent polymers that were simply sprinkled from above the substrate sheet and are not fixed with an adhesive or the like). In the operation of suspending the measurement sample, it is sufficient that the measurement sample is substantially vertically suspended for 3 to 5 seconds simply by holding an end portion of the measurement sample. It is unnecessary to hit or significantly shake the suspended measurement sample. Also, when suspending the measurement sample, first, an end portion of the measurement sample is held using a pair of tweezers or the like, and the measurement sample is suspended for 3 to 5 seconds. After that, an end portion that is opposite to the end portion that was held to suspend the measurement sample is held to suspend the measurement sample for 3 to 5 seconds.

Also, in step 1, in the case where an absorbent sheet with a 5 cm×5 cm square shape cannot be prepared as a measurement sample (for example, in the case where the absorbent sheet is too small in size that the shape of the measurement sample does not satisfy a 5 cm×5 cm square), a plurality of measurement samples, each having a size that does not satisfy a 5 cm×5 cm square, are obtained from a sheet prepared as an evaluation target, and the plurality of measurement samples are arranged such that the total area of one surfaces of the plurality of measurement samples is 25 cm². Then, for each of the plurality of measurement samples, the water-absorbent polymer fixation rate is measured through steps 1 to 5 described above, and the average value of a plurality of water-absorbent polymer fixation rates thus obtained is defined as the water-absorbent polymer fixation rate of the absorbent sheet.

Examples of the measurement equipment used in step 3 are as follows.
Magnetic stirrer: HI-304N (reverse rotation stirrer available from HANNA Instruments)
Stirring bar: star-headed NALGENE (6600-0035) [35 φ×12 mm]
Beaker: 300 ml [78 φ×103 mm]

There is no particular limitation on the fixing means used to fix the water-absorbent polymers P1 to the substrate layer 20. In short, any fixing means can be used as long as the water-absorbent polymers P1 can be reliably fixed to the substrate layer 20, and detachment of the water-absorbent polymers P1 can be effectively prevented not only when the water-absorbent polymers P1 have not absorbed a liquid, but also when the water-absorbent polymers P1 have absorbed a liquid and are swollen. Preferably, as described above, the fixing means has a fixation rate of the water-absorbent polymers P1 to the substrate layer 20 of 10% or more. A typical example of the fixing means is an adhesive. The adhesive used herein encompasses both “an adhesive in a limited sense that is a liquid (fluid) before use and is a solid when attached to an adherend” and “a pressure-sensitive adhesive that has both properties of a liquid and a solid and constantly keeps a wet state in a stable manner”. In the case where a fixing means other than an adhesive is used, the water-absorbent polymers P1 may be fixed to the substrate layer 20 by, for example, causing the water-absorbent polymers P1 to directly adhere to the surface of the substrate layer 20 without any member such as an adhesive being interposed therebetween. The water-absorbent polymers P1 directly adhered to the surface of the substrate layer 20 can be obtained by producing water-absorbent polymers on the substrate layer 20, specifically, performing a polymerization reaction of water-absorbent polymers on the substrate layer 20.

In the present embodiment, the water-absorbent polymers P1 of the absorbent layer 21 are fixed to one side of the substrate layer 20 using a hot melt adhesive (not shown). By using a hot melt adhesive as an adhesive for fixing the water-absorbent polymers P1 to substrate sheets, the water-absorbent polymers P1 can be fixed to the substrate layer 20 while ensuring the flexibility of the substrate layer 20, more preferably, a fixation rate of the water-absorbent polymers P1 to the substrate layer 20 of 10% or more can be achieved, and also gaps are unlikely to occur between the absorbent layer 21 (the water-absorbent polymers P1) and the entangled fiber layer 3, and thus the effect of keeping the entangled fiber layer 3 from breaking apart can be further improved.

As the hot melt adhesive used as the adhesive for fixing the water-absorbent polymers P1 to substrate sheets, it is preferable to use a flexible hot melt adhesive that can stretch along with changes in the water-absorbent polymers P1 that absorb a liquid and swell. Specifically, it is preferable to use an acrylic hot melt adhesive, a silicone-based hot melt adhesive, a rubber-based hot melt adhesive, or an olefin-based hot melt adhesive. As an index for flexibility of the adhesive, the maximum elongation (elongation at break) of the adhesive measured in accordance with Japan Adhesive Industry Association Standard JAI7-1999 can be used. The higher the value of the maximum elongation, the higher the flexibility of the adhesive. The maximum elongation of the hot melt adhesive is preferably 200% or more, and more preferably 300% or more. It is more preferable that the value of the maximum elongation of the hot melt adhesive, or in other words, the flexibility of the hot melt adhesive is higher. However, the upper limit for maximum elongation is preferably 3000% or less, and more preferably 2500% or less.

Examples of the material (base polymer) of the acrylic adhesive include those composed of (co)polymers of vinyl monomers (an ethylene vinyl acetate copolymer and the like) in which 2-ethylhexyl acrylate, butyl acrylate, ethyl acrylate, cyano acrylate, vinyl acetate, methyl methacrylate, and the like are used as main components.

An example of the material (base polymer) of the silicone-based adhesive includes a polydimethyl siloxane polymer.

Examples of the material (base polymer) of the rubber-based adhesive include natural rubber, polyisoprene, a styrene-butadiene copolymer (SBR), a styrene-isoprene-styrene block copolymer (SIS), a styrene-butadiene-styrene block copolymer (SBS), a styrene-ethylene-butadiene-styrene block copolymer (SEBS), and a styrene-ethylene-propylene-styrene block copolymer (SEPS).

As the substrate layer 20, any sheet can be used as long as the water-absorbent polymers P1 can be fixed thereto. The sheet may be a liquid-permeable sheet or a liquid-impermeable sheet. Examples of the substrate layer 20 include: fiber structures such as a non-woven fabric, a woven fabric, a knitted fabric, and paper; as well as a resin film, a foam, a net, and the like. In particular, from the viewpoint of reducing the thickness of the absorbent sheet 2 and improving flexibility, the substrate layer 20 preferably includes a non-woven fabric or a resin film, or in other words, the substrate layer 20 preferably includes either one of a non-woven fabric and a resin film, or is preferably a composite sheet that includes both a non-woven fabric and a resin film.

As the non-woven fabric that constitutes the substrate layer 20, a non-woven fabric produced using any production method can be used without any particular limitation. Examples of the non-woven fabric include an air-through non-woven fabric, a heat roll non-woven fabric, a spun-lace non-woven fabric, a spun-bond non-woven fabric, a melt-blown non-woven fabric, and a spun-bond melt-blown spun-bond (SMS) non-woven fabric. These non-woven fabrics may be hydrophilic non-woven fabrics composed of fibers that have undergone hydrophilization. Out of these non-woven fabrics, in particular, a SMS non-woven fabric is preferably used as the substrate layer 20 because it is particularly effective for reducing the thickness of the absorbent sheet 2 and improving flexibility.

Examples of the material of the resin film that constitutes the substrate layer 20 include polyurethane resins, polyester-based resins such as polyethylene terephthalate (PET), polyolefin-based resins such as polyethylene (PE) and polypropylene (PP), polyamide resins, polyvinyl alcohols, and modified products and copolymers thereof. From the viewpoint of improving liquid permeability, and the like, a plurality of holes penetrating in the thickness direction may be formed in the resin film. Out of these resin films, in particular, a polyolefin-based resin is preferably used for the substrate layer 20 because it has excellent flexibility and processability.

The absorbent sheet 2 can be produced by, for example, applying an adhesive (preferably, a hot melt adhesive) onto one side of a substrate layer 20, and thereafter spraying water-absorbent polymers P1 (water-absorbent polymer pieces) on the side of the substrate layer 20 on which the adhesive has been applied. There is no particular limitation on the application pattern of the adhesive. The adhesive may be applied to the entirety of or a portion of one side of the substrate layer 20. In the case where, for example, the absorbent sheet 2 is used as an absorbent member for an absorbent article, from the viewpoint of minimizing a reduction in absorbency caused by the adhesive, the adhesive is preferably applied to a portion, or in other words, the adhesive is preferably applied such that both an adhesive applied portion and an adhesive non-applied portion are present on one side of the substrate layer 20. There is no particular limitation on the method for applying an adhesive, and any known application method can be used such as, for example, a slot spray method, a curtain spray method, a spiral spray method, a coater spray method, an omega spray method, or a summit spray method. The amount of adhesive applied is preferably 1 g/m² or more, and more preferably 5 g/m² or more, and preferably 30 g/m² or less, and more preferably 20 g/m² or less in terms of solid content.

There is no particular limitation on the thickness of the substrate layer 20, the basis weight of the substrate layer 20, and the basis weight of the water-absorbent polymers P1 (the amount of water-absorbent polymers P1 attached per unit area). Appropriate values can be selected according to specific applications of the absorbent member 1, and the like. In the case where the absorbent member 1 is used as, for example, an absorbent member for an absorbent article, the absorbent member 1 is to be thin irrespective of the basis weight of the water-absorbent polymers P1 being relatively high. Specifically, in the case where the absorbent member 1 is used as an absorbent member for an absorbent article, from the viewpoint of obtaining a thin and flexible absorbent member that has a sufficient absorbent capacity, the basis weight of the water-absorbent polymers P1 is preferably 30 g/m² or more, and more preferably 100 g/m² or more, and preferably 600 g/m² or less, and more preferably 400 g/m² or less. In the present embodiment, the absorbent layer 21 is composed only of water-absorbent polymers P1, but may contain a component other than water-absorbent polymers such as fibers, without departing from the gist of the present invention.

From the same viewpoint, the thickness (substantial thickness) of the substrate layer 20 is preferably 0.01 mm or more, more preferably 0.03 mm or more, and preferably 0.8 mm or less, and more preferably 0.2 mm or less. In the description of the present application, unless otherwise stated, the term “sheet thickness” refers to a thickness measured using the following method (a thickness under a load of 0.5 cN/cm² (=0.05 kPa)).

From the same viewpoint, the basis weight of the substrate layer 20 is preferably 5 g/m² or more, and more preferably 8 g/m² or more, and preferably 40 g/m² or less, and more preferably 25 g/m² or less.

### Method for Measuring Sheet Thickness

A circular plate with a weight of 2.5 g and a radius of 12.5 mm is placed on a measurement table, and a position on the upper surface of the circular plate in that state is defined as measurement reference point A. Next, the circular plate is removed, and a measurement target is placed on the measurement table. Then, the circular plate is again placed thereon, and a position on the upper surface of the circular plate in that state is defined as position B. As the measurement equipment, a laser displacement meter (CCD laser displacement sensor LK-080 available from Keyence Corporation) is used. The difference between the reference point A and the position B is defined as the thickness of the measurement target, or in other words, the thickness of the measurement target under a pressure of 0.5 cN/cm² (=0.05 kPa).

In the case where the measurement target sheet (for example, the substrate layer 20) is incorporated in an absorbent article, the measurement target sheet can be taken out from the absorbent article in the following manner. Cold Spray (product name, available from Nichiban Co., Ltd.) is sprayed from the top sheet side of the absorbent article. After that, the constituent members that constitute the absorbent article other than the measurement target are carefully separated from the absorbent article.

As schematically shown in FIGS. 1 and 2, the entangled fiber layer 3 is a fiber assembly in which a plurality of fibers 3F are entangled with each other without being fused to each other. The entangled fiber layer 3 functions to compensate for insufficient absorbency (absorbent capacity, absorption speed, and the like) of the thin absorbent sheet 2.

The entangled fiber layer of the present invention is not a layer obtained through wet papermaking. Accordingly, the entangled fiber layer 3 that is an embodiment of the entangled fiber layer of the present invention does not encompass paper produced through a known wet papermaking method. There is no particular limitation on the type of fibers 3F that constitute the entangled fiber layer 3, and fibers that are widely used to make paper, for example, pulp fibers can be used as the fibers 3F. If the entangled fiber layer 3 is, for example, a sheet obtained through wet papermaking such as a sheet of paper, the surface of the entangled fiber layer 3 is too smooth, or the number of empty spaces (empty spaces between fibers) in the entangled fiber layer 3 is too small, and thus the contact between the entangled fiber layer 3 and the water-absorbent polymers P1 of the absorbent layer 21 is reduced, and the effects of the present invention may not be exhibited. That is, as the entangled fiber layer 3, it is preferable to use a fiber assembly that has a lower surface smoothness and includes a larger number of empty spaces between fibers, as compared with a sheet obtained through wet papermaking such as a sheet of paper.

Also, in the entangled fiber layer 3, the plurality of fibers 3F are entangled with each other, but are not fused to each other. That is, in the entangled fiber layer 3, there are no bonding points formed by fusion of fibers 3F. Accordingly, the entangled fiber layer 3 does not encompass, for example, a resin bond non-woven fabric in which constituent fibers are thermally fused to each other. As used herein, the bonding points of fibers 3F refers to bonding points between constituent fibers that are inherently included in the entangled fiber layer 3, and bonding points that are formed as a result of post-processing that involves melting constituent fibers 3F being performed on the entangled fiber layer 3 are excluded. A typical example of the bonding points of fibers 3F may be bonding points of fibers 3F that are formed through hot air processing when a non-woven fabric used as the entangled fiber layer 3 is produced by performing hot air processing using an air-through method on a web produced according to a conventional method using a carding machine. On the other hand, portions (for example, filmed portions) that are formed as a result of emboss processing that uses heat being performed on the entangled fiber layer 3 as post-processing are not included in the bonding points defined herein.

An entangled fiber layer 3 that does not include bonding points formed by fusion of fibers 3F is typically a fiber assembly (sheet-like member) other than a non-woven fabric. Specific examples include, a web produced according to a conventional method using a carding method or an air-laid method, and a fiber stack of pulp defibrated from a pulp sheet, but layers obtained through wet papermaking are excluded as described above. With the entangled fiber layer 3 that does not include bonding points formed by fusion of fibers 3F, as compared with the same that includes bonding points formed by fusion of fibers 3F, the flexibility of the entangled fiber layer 3 is improved. Also, when an absorbent member 1 that includes the entangled fiber layer 3 is used in an absorbent article, it may lead to an improvement in the wearing comfort of the absorbent article. Also, in the present embodiment, as shown in FIGS. 1 and 2, the entangled fiber layer 3 contains water-absorbent polymers P2. When the entangled fiber layer 3 includes bonding points formed by fusion of fibers 3F, swelling of the water-absorbent polymers P2 after absorbing a liquid is impaired, which may lead to a reduction in the absorbency of the absorbent member 1.

From the viewpoint of improving the absorbency for absorbing a body fluid, the entangled fiber layer 3 preferably contains pulp fibers (cellulose fibers). That is, it is preferable that at least some of the constituent fibers 3F of the entangled fiber layer 3 are pulp fibers. In general, the fiber length of pulp fibers is short, and thus the entangled fiber layer 3 composed mainly of pulp fibers has insufficient shape stability when it is wet and is likely to break apart in a case where an external force is applied when it is wet. However, as described above, in the absorbent member 1, the entangled fiber layer 3 is disposed adjacent to the absorbent layer 21 (the water-absorbent polymers P1) of the absorbent sheet 2, and thus even when the entangled fiber layer 3 is composed mainly of pulp fibers, breaking apart of the entangled fiber layer 3 is effectively suppressed.

As the pulp fibers, any pulp fibers that are conventionally used in this type of absorbent member can be used without any particular limitation. Examples include: natural fibers including wood pulps such as softwood pulp and hardwood pulp and non-wood pulps such as cotton pulp and hemp pulp; modified pulps such as cationized pulp and mercerized pulp; and the like. These may be used alone or in a combination of two or more. The amount of pulp fibers contained in the entangled fiber layer 3 is preferably 30 mass % or more, and more preferably 50 mass % or more relative to the total mass of the entangled fiber layer 3, and may be 100 mass %, or in other words, the entangled fiber layer 3 may be composed only of pulp fibers.

The entangled fiber layer 3 may have a monolayer structure or a multilayer structure that includes a plurality of layers with different compositions such as a multilayer structure that includes a plurality of layers that are composed of different types of constituent fibers 3F. In the present embodiment, the entangled fiber layer 3 has a monolayer structure.

From the viewpoint of improving the absorbency for absorbing a body fluid, the entangled fiber layer 3 may contain water-absorbent polymers. In the present embodiment, as shown in FIGS. 1 and 2, the entangled fiber layer 3 contains water-absorbent polymers P2 in addition to the fibers 3F. When the entangled fiber layer 3 contains water-absorbent polymers P2, entanglement between fibers 3F is hindered by the water-absorbent polymers P2, and thus the above-described breaking apart of the entangled fiber layer 3 is likely to occur. However, as described above, in the absorbent member 1, the entangled fiber layer 3 is disposed adjacent to the absorbent layer 21 (the water-absorbent polymers P1) of the absorbent sheet 2, and thus even when the entangled fiber layer 3 contains water-absorbent polymers P2, breaking apart of the entangled fiber layer 3 is effectively suppressed. The content of water-absorbent polymers P2 in the entangled fiber layer 3 is preferably 20 mass % or more, more preferably 50 mass % or more, and preferably 80 mass % or less, and more preferably 70 mass % or less relative to the total mass of the entangled fiber layer 3.

As the water-absorbent polymers P2 of the entangled fiber layer 3, any water-absorbent polymers that can be used as the water-absorbent polymers P1 of the absorbent layer 21 can be used. The type, shape, and average particle size of water-absorbent polymers may be the same or different in the water-absorbent polymers P1 and the water-absorbent polymers P2.

From the viewpoint of improving the absorbency for absorbing a body fluid, in the case where the entangled fiber layer 3 contains water-absorbent polymers P2, the content of water-absorbent polymers P2 in the entangled fiber layer 3 (hereinafter also referred to as “water-absorbent polymer content R3P”) is preferably higher than the content of fibers 3F in the entangled fiber layer 3 (hereinafter also referred to as “fiber content R3F”). The water-absorbent polymer content R3P is calculated by dividing the total weight of water-absorbent polymers P2 contained in the entangled fiber layer 3 by the weight of the entangled fiber layer 3, and the fiber content R3F is calculated by dividing the total weight of constituent fibers 3F that constitute the entangled fiber layer 3 by the weight of the entangled fiber layer 3.

In the entangled fiber layer 3, the ratio of the water-absorbent polymer content R3P to the fiber content R3F, which is represented by R3P/R3F, is preferably 1.1 or more, and more preferably 1.3 or more, and preferably 3.5 or less, and more preferably 2.5 or less, on the assumption that R3P>R3F as described above.

The water-absorbent polymer content R3P in the entangled fiber layer 3 is preferably 55 mass % or more, and more preferably 60 mass % or more, and preferably 80 mass % or less, and more preferably 70 mass % or less.

The fiber content R3F in the entangled fiber layer 3 is preferably 20 mass % or more, and more preferably 30 mass % or more, and preferably 45 mass % or less, and more preferably 40 mass % or less.

In the case where the entangled fiber layer 3 contains water-absorbent polymers P2 as in the present embodiment, it is preferable that the water-absorbent polymers P2 of the entangled fiber layer 3 are embedded in the absorbent layer 21. With this configuration, the water-absorbent polymers P1 (the absorbent layer 21) and the entangled fiber layer 3 are more strongly bonded, and thus the above-described effect of keeping the entangled fiber layer 3 from breaking apart can be further improved.

Whether the water-absorbent polymers P2 are embedded in the absorbent layer 21 can be determined using the following method. Specifically, a cross section of the absorbent member 1 in the thickness direction as shown in FIG. 2 is observed using an electron microscope. Then, in the observed image, the number of water-absorbent polymers P2 embedded in the absorbent layer 21 is counted. If the number of water-absorbent polymers P2 embedded in the absorbent layer 21 is greater than or equal to a predetermined reference value, it is determined that the water-absorbent polymers P2 are embedded in the absorbent layer 21. More specifically, it is determined that the water-absorbent polymers P2 are embedded in the absorbent layer 21 if, in the observed image captured by the electron microscope, there is at least one group of water-absorbent polymers P2 per 5 mm width, the group of water-absorbent polymers P2 including at least twenty water-absorbent polymers P2 of the entangled fiber layer 3 whose lower ends (end portions on an adjacent surface side that is adjacent to the absorbent layer 21) extend to positions lower than the upper ends (end portions on an entangled fiber layer 3 side) of the water-absorbent polymers P1 of the absorbent layer 21.

Also, from the viewpoint of improving the effect of keeping the entangled fiber layer 3 from breaking apart, in the case where the entangled fiber layer 3 contains water-absorbent polymers P2 as in the present embodiment, it is preferable that, in the entangled fiber layer 3, the content of fibers 3F is higher on the adjacent surface side that is adjacent to the absorbent layer 21 (the water-absorbent polymers P1) than on the side opposite to the adjacent surface. More specifically, as shown in FIG. 2, where one of two equal parts into which the entangled fiber layer 3 that contains fibers 3F and water-absorbent polymers P2 is divided in the thickness direction (the up-down direction in FIG. 2) that is relatively closer to the absorbent layer 21 is denoted as an absorbent layer side 30, and the other one of the two equal parts that is on the opposite side is denoted as a non-absorbent layer side 31, the content of fibers 3F is preferably set such that the following relationship is satisfied: absorbent layer side 30>non-absorbent layer side 31. In other words, in the entangled fiber layer 3, it is preferable that the constituent fibers 3F are distributed concentratedly on the absorbent layer side 30, and the water-absorbent polymers P2 are distributed concentratedly on the non-absorbent layer side 31. As described above, when the absorbent layer side 30 (the adjacent surface side that is adjacent to the absorbent layer 21) of the entangled fiber layer 3 is a “constituent fiber-rich layer” that contains a larger amount of fibers 3F than that on the non-absorbent layer side 31, the constituent fibers 3F of the absorbent layer side 30 and the absorbent layer 21 (the water-absorbent polymers P1) are more strongly bonded, and thus the above-described effect of keeping the entangled fiber layer 3 from breaking apart can be further improved.

With respect to the content of constituent fibers 3F in each portion of the entangled fiber layer 3, it is preferable to set the content of constituent fibers 3F in each portion of the entangled fiber layer 3 in a manner described below on the assumption that the above-described relationship “absorbent layer side 30>non-absorbent layer side 31” is satisfied.

The content of fibers 3F in the absorbent layer side 30 (the adjacent surface side that is adjacent to the absorbent layer 21) is preferably 30 mass % or more, and more preferably 40 mass % or more, and preferably 100 mass % or less, and more preferably 90 mass % or less relative to the total mass of the absorbent layer side 30.

The content of fibers 3F in the non-absorbent layer side 31 is preferably 10 mass % or more, and more preferably 20 mass % or more, and preferably 80 mass % or less, and more preferably 70 mass % or less relative to the total mass of the non-absorbent layer side 31.

From the same viewpoint, in the absorbent member 1, it is preferable that the content of fibers is higher in the following order: 1) the absorbent layer side 30 (the adjacent surface side that is adjacent to the absorbent layer 21) of the entangled fiber layer 3; 2) the non-absorbent layer side 31 of the entangled fiber layer 3; and 3) the absorbent layer 21. That is, it is preferable that the content of fibers in the absorbent layer side 30 of the entangled fiber layer 3 is the highest, the content of fibers in the absorbent layer 21 is the lowest, and the content of fibers in the non-absorbent layer side 31 of the entangled fiber layer 3 falls in the range between the highest and lowest values. As used herein, the term “fibers” used in the expression “the content of fibers” encompasses not only the constituent fibers 3F of the entangled fiber layer 3, but also all of the fibers contained in the absorbent member 1. However, usually, the constituent fibers 3F of the entangled fiber layer 3 are the main component of “fibers”. As described above, because the content of fibers in the absorbent member 1 varies from portion to portion, the above-described effect of keeping the entangled fiber layer 3 from breaking apart is further improved. Also, in the case where the absorbent member 1 is used in an absorbent article, usually, the entangled fiber layer 3 is disposed closer to the skin of the wearer than the absorbent sheet 2 is, and thus, in the entangled fiber layer 3, the non-absorbent layer side 31 is relatively close to the skin of the wearer, and the absorbent layer side 30 is relatively far from the skin of the wearer. However, with the configuration in which the non-absorbent layer side 31 that is relatively closer to the skin of the wearer has a fiber content lower than the absorbent layer side 30 that is relatively far from the skin of the wearer, so-called backflow, which is a flowing back of a liquid once absorbed bythe absorbent layer 21 (the water-absorbent polymers P1) toward the skin of the wearer, can be effectively suppressed.

With respect to the content of fibers in each portion of the absorbent member 1, on the assumption that the above-described relationship “absorbent layer side 30 of entangled fiber layer 3>non-absorbent layer side 31 of entangled fiber layer 3>absorbent layer 21” is satisfied, the content of fibers in the absorbent layer side 30 and the content of fibers in the non-absorbent layer side 31 can be set in the above-described ranges. The content of fibers in the absorbent layer 21 is preferably 30 mass % or less, and more preferably 10 mass % or less relative to the total mass of the absorbent layer 21, and may be zero, or in other words, the absorbent layer 21 may contain no fibers.

There is no particular limitation on the basis weight of the entangled fiber layer 3, but from the viewpoint of improving wearing comfort, portability, and the like in the case where the absorbent member 1 is used in an absorbent article while the absorbent member 1 is kept thin, the basis weight is preferably as small as possible while ensuring sufficient absorbency required for the absorbent member 1. From this viewpoint, the basis weight of fibers in the entangled fiber layer 3 is preferably 50 g/m² or more, and more preferably 90 g/m² or more, and preferably 200 g/m² or less, and more preferably 170 g/m² or less. As used herein, the expression “the basis weight of fibers in the entangled fiber layer 3” means, in the case where the entangled fiber layer 3 has a stack structure in which a plurality of layers are stacked, the basis weight of fibers in the entire stack structure.

The absorbent member 1 can be used alone to absorb a liquid. For example, the absorbent member 1 can be used as a drip absorbent sheet, a sheet placed under a food product, a sheet used in a litter box for pet animals, or the like. It can also be used as an absorbent member in a sanitary product such as a medical pad or a breast-milk absorbent sheet. Particularly preferably, the absorbent member 1 is used as an absorbent member in an absorbent article such as a sanitary napkin or a disposable diaper.

The present invention encompasses an absorbent article that includes the above-described absorbent member of the present invention. The absorbent article according to the present invention typically includes: a top sheet that is positioned close to the skin of a wearer; a back sheet that is positioned far from the skin of the wearer; and the absorbent member of the present invention, specifically, for example, the absorbent member 1 described above, that is provided between the top sheet and the back sheet. In this case, the absorbent member 1 may be disposed such that the entangled fiber layer 3 is closer to the skin of the wearer (the top sheet) than the absorbent sheet 2 is, or may be disposed such that the absorbent sheet 2 is closer to the skin of the wearer (the top sheet) than the entangled fiber layer 3 is. However, from the viewpoint of improving the wearing comfort and cushioning of the absorbent article, the former arrangement in which the entangled fiber layer 3 is provided on the skin side (top sheet side) and the absorbent sheet 2 is provided on the non-skin side (back sheet side) is preferable. In the case where the absorbent member 1 is provided in an absorbent article such that the entangled fiber layer 3 is positioned on the skin side, a disadvantage may occur in that the entangled fiber layer 3 is more likely to be subjected to an external force due to movement of the wearer and break apart, as compared with the case where the absorbent member 1 is provided such that the entangled fiber layer 3 is positioned on the non-skin side. However, with the above-described characteristic configuration of the absorbent member 1, that disadvantage is eliminated. The absorbent article according to the present invention includes the absorbent member of the present invention such as the absorbent member 1. Accordingly, the absorbent article according to the present invention has excellent absorbency and shape stability while being thin, and the disadvantage of the absorbent member breaking apart when the absorbent article is worn is unlikely to occur.

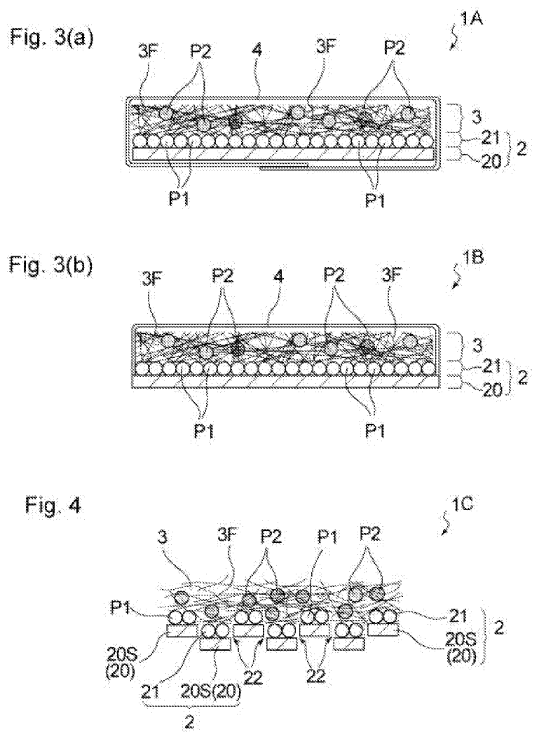

FIGS. 3 and 4 show other embodiments of the absorbent member according to the present invention. In the following description of other embodiments, constituent members that are different from those of the embodiment given above (the absorbent member 1) will be mainly described, and constituent members that are the same as those of the embodiment given above (the absorbent member 1) will be given the same reference numerals, and a description thereof will be omitted. Regarding the constituent elements not described below, see the description of the embodiment given above as appropriate.

Absorbent members 1A and 1B shown in FIG. 3 each include a cover sheet 4 that integrally covers the absorbent sheet 2 and the entangled fiber layer 3. Due to the presence of the cover sheet 4 that covers the absorbent sheet 2 and the outer surface of the entangled fiber layer 3, the adhesion between the absorbent sheet 2 and the entangled fiber layer 3 increases, and thus the water-absorbent polymers P1 of the absorbent layer 21 are more strongly bonded to the entangled fiber layer 3. As a result, the effect of keeping the entangled fiber layer 3 from breaking apart can be further improved. As the cover sheet 4, a water permeable sheet material can be used such as, for example, paper or a non-woven fabric. The basis weight of the cover sheet 4 is preferably 8 g/m² or more, and more preferably 12 g/m² or more, and preferably 30 g/m² or less, and more preferably 20 g/m² or less.

The cover sheet 4 of the absorbent member 1A is, as shown in FIG. 3(a), one continuous sheet with a size that can cover the entire outer surface of the stack structure that includes the absorbent sheet 2 and the entangled fiber layer 3, and covers the entire region of the surface of the entangled fiber layer 3 (for example, skin-facing surface in the case where the absorbent member 1A is used in an absorbent article) and the entire region of the surface of the absorbent sheet 2 (for example, non-skin-facing surface in the case where the absorbent member 1A is used in an absorbent article). The absorbent sheet 2, the entangled fiber layer 3, and the cover sheet 4 may be bonded to each other using an adhesive. The cover sheet 4 of the absorbent member 1A does not need to be one sheet as shown in the diagram, and may be composed of, for example, two layers: a skin side cover sheet that covers the surface of the entangled fiber layer 3; and a non-skin side cover sheet that is provided separately from the skin side cover sheet and covers the surface of the absorbent sheet 2.

In the description of the present application, the term “skin-facing surface” refers to a surface of an absorbent article or its constituent member (for example, absorbent member) that is provided to face the skin of the wearer when the absorbent article is worn, or in other words, a surface that is relatively close to the skin of the wearer, and the term “non-skin-facing surface” refers to a surface of an absorbent article or its constituent member that is provided to face the side opposite to the skin of the wearer when the absorbent article is worn (garment side), or in other words, a surface that is relatively far from the skin of the wearer. As used herein, the expression “when an absorbent article is worn” means a state in which the normal and correctly worn position of the absorbent article is maintained.

As shown in FIG. 3(b), the cover sheet 4 of the absorbent member 1B covers the entire region of the surface of a portion (the absorbent layer 21 and the entangled fiber layer 3) other than the substrate layer 20 in the stack structure that includes the absorbent sheet 2 and the entangled fiber layer 3, and does not cover the substrate layer 20. In the absorbent member 1B, the peripheral portion of the cover sheet 4 and the peripheral portion of the substrate layer 20 are bonded to each other using an adhesive or the like to form a space defined by the cover sheet 4 and the substrate layer 20, and the absorbent layer 21 and the entangled fiber layer 3 are incorporated in the space.

In an absorbent member 1C shown in FIG. 4, the substrate layer 20 includes a plurality of (seven in FIG. 4) strip-like substrate layers 20S that are elongated in one direction. The plurality of strip-like substrate layers 20S are disposed such that their lengthwise directions match, and the plurality of strip-like substrate layers 20S are adjacent to each other in a width direction that is perpendicular to the lengthwise direction. As shown in FIG. 4, the plurality of strip-like substrate layers 20S that are adjacent to each other in the width direction are divided by gaps (slits) 22 that penetrate through the substrate layer 20 in the thickness direction. In the absorbent member 1C configured as described above, the plurality of strip-like substrate layers 20S are independently movable. Accordingly, for example, as shown in FIG. 4, the plurality of strip-like substrate layers 20S may be at different positions in the thickness direction of the absorbent member 1C.

The surface of the entangled fiber layer 3 may have irregularities due to uneven texture or the like. If there are irregularities in the adjacent surface that is adjacent to the absorbent layer 21, the adhesion between the absorbent layer 21 (the water-absorbent polymers P1) and the entangled fiber layer 3 decreases, and the above-described effect of keeping the entangled fiber layer 3 from breaking apart may be reduced. However, in the absorbent member 1C, as described above, the substrate layer 20 includes a plurality of (seven in FIG. 4) strip-like substrate layers 20S that are independently movable. Accordingly, even if there are some irregularities in the adjacent surface of the entangled fiber layer 3 that is adjacent to the absorbent layer 21, the plurality of strip-like substrate layers 20S that constitute the substrate layer 20 can follow the irregularities of the entangled fiber layer 3 and adhere to the entangled fiber layer 3. Accordingly, the expected effect of keeping the entangled fiber layer 3 from breaking apart is exhibited in a stable manner.

In the absorbent member 1C, there is no particular limitation on the extension direction of the gaps 22 of the substrate layer 20. However, from the viewpoint of more reliably exhibiting the advantageous effect of the gaps 22 (strip-like substrate layers 20S), the extension direction of the gaps 22 of the substrate layer 20 is preferably set such that it matches the longitudinal direction of the absorbent article in which the absorbent member 1C is used. As used herein, the longitudinal direction of the absorbent article refers to the front-back direction of the wearer of the absorbent article, or in other words, the direction that extends from the abdominal side to the back side of the wearer via a crotch portion.

Also, from the viewpoint of improving the shape stability of the substrate layer 20, it is preferable that the plurality of strip-like substrate layers 20S are integrated without being divided. For example, although not shown in the diagram, it is preferable that the end portions of the plurality of strip-like substrate layers 20S in the lengthwise direction are connected without being divided. In the absorbent member 1C according to the preferred embodiment, in a portion between the end portions of the plurality of strip-like substrate layers 20S in the lengthwise direction of the substrate layer 20, a plurality of gaps 22 that extend in the lengthwise direction are intermittently provided in the width direction of the substrate layer 20, as a result of which the portion between the end portions of the plurality of strip-like substrate layers 20S in the lengthwise direction is divided into the plurality of strip-like substrate layers 20S. However, the plurality of strip-like substrate layers 20S are integrated at their end portions in the lengthwise direction of the substrate layer 20, and thus the absorbent member 1C is deemed as one object as a whole.

Up to here, the present invention has been described by way of embodiments of the present invention. However, the present invention is not limited to the embodiments described above, and can be modified as appropriate.

For example, in the embodiments given above, the absorbent layer 21 is formed only on one side of the substrate layer 20. However, the absorbent layer 21 may be formed on each side of the substrate layer 20. That is, the water-absorbent polymers P1 may be fixed to both sides of the substrate layer 20. In particular, in the absorbent member 1C shown in FIG. 4, when the absorbent layer 21 is formed on both sides of the substrate layer 20, it is possible to obtain an effect in which the contact between the water-absorbent polymers P1 and the entangled fiber layer 3 is maintained even if the strip-like substrate layers 20S are twisted and reversed.

Also, an absorbent article in which the absorbent member of the present invention is used, as well as the absorbent article according to the present invention, broadly encompasses articles that are used to absorb a body fluid (urine, soft feces, menstrual blood, sweat, and the like) discharged from a human body, such as, for example, an open-type disposable diaper, a pant-type disposable diaper, a sanitary napkin, sanitary pants. With respect to the embodiments of the present invention described above, the following additional statements are also disclosed.

<1> An absorbent member comprising:

an absorbent sheet that includes a substrate layer and an absorbent layer that includes water-absorbent polymers that are fixed to one side of the substrate layer; and

an entangled fiber layer,

wherein the water-absorbent polymers of the absorbent layer and the entangled fiber layer are adjacent to each other.

<2> The absorbent member as set forth in clause <1>,

wherein the water-absorbent polymers of the absorbent layer are fixed to the one side of the substrate layer using a hot melt adhesive.

<3> The absorbent member as set forth in clause <1> or <2>,

wherein the substrate layer includes a non-woven fabric or a resin film.

<4> The absorbent member as set forth in any one of clauses <1> to <3>,

wherein the entangled fiber layer includes pulp fibers.

<5> The absorbent member as set forth in any one of clauses <1> to <4>,

wherein the entangled fiber layer includes water-absorbent polymers.

<6> The absorbent member as set forth in clause <5>,

wherein the content of the water-absorbent polymers in the entangled fiber layer is preferably 20 mass % or more, more preferably 50 mass % or more, and preferably 80 mass % or less, and more preferably 70 mass % or less relative to the total mass of the entangled fiber layer.

<7> The absorbent member as set forth in clause <5> or <6>,

wherein the content of the water-absorbent polymers in the entangled fiber layer is higher than the content of the fibers in the entangled fiber layer.

<8> The absorbent member as set forth in clause <7>,

wherein the ratio between the content of the water-absorbent polymers in the entangled fiber layer and the content of the fibers in the entangled fiber layer, which is represented by the former/the latter, is preferably 1.1 or more, and more preferably 1.3 or more, and preferably 3.5 or less, and more preferably 2.5 or less, on the assumption that the former>the latter.

<9> The absorbent member as set forth in clause <7> or <8>,

wherein the content of the water-absorbent polymers in the entangled fiber layer is preferably 55 mass % or more, and more preferably 60 mass % or more, and preferably 80 mass % or less, and more preferably 70 mass % or less.

<10> The absorbent member as set forth in any one of clauses <7> to <9>,

wherein the content of the fibers in the entangled fiber layer is preferably 20 mass % or more, and more preferably 30 mass % or more, and preferably 45 mass % or less, and more preferably 40 mass % or less.

<11> The absorbent member as set forth in any one of clauses <5> to <10>,

wherein the water-absorbent polymers of the entangled fiber layer are embedded in the absorbent layer.

<12> The absorbent member as set forth in any one of clauses <5> to <11>,

wherein, in the entangled fiber layer, the content of the fibers is higher on an adjacent surface side where an adjacent surface that is adjacent to the absorbent layer (absorbent layer side) is present than on a side opposite to the adjacent surface (non-absorbent layer side).

<13> The absorbent member as set forth in any one of clauses <5> to <12>,

wherein, in the entangled fiber layer, the fibers are distributed concentratedly on an adjacent surface side where an adjacent surface that is adjacent to the absorbent layer (absorbent layer side) is present, and the water-absorbent polymers are distributed concentratedly on a side that is opposite to the side where the absorbent layer is present.

<14> The absorbent member as set forth in any one of clauses <1> to <13>,

wherein the content of the fibers in the entangled fiber layer on an adjacent surface side where an adjacent surface that is adjacent to the absorbent layer (absorbent layer side) is present is preferably 30 mass % or more, and more preferably 40 mass % or more, and preferably 100 mass % or less, and more preferably 90 mass % or less relative to the total mass of the adjacent surface side.

<15> The absorbent member as set forth in any one of clauses <1> to <14>,

wherein the content of the fibers in the entangled fiber layer on an opposite side that is opposite to the side where the absorbent layer (non-absorbent layer side) is present is preferably 10 mass % or more, and more preferably 20 mass % or more, and preferably 80 mass % or less, and more preferably 70 mass % or less relative to the total mass of the opposite side.

<16> The absorbent member as set forth in any one of clauses <1> to <15>,

wherein the basis weight of the fibers in the entangled fiber layer is preferably 50 g/m² or more, and more preferably 90 g/m² or more, and preferably 200 g/m² or less, and more preferably 170 g/m² or less.

<17> The absorbent member as set forth in any one of clauses <1> to <16>,

wherein the entangled fiber layer does not include a bonding point formed by fusion of fibers, and is a web produced using a carding method or an air-laid method, or a fiber stack of fibers defibrated from a pulp sheet.

<18> The absorbent member as set forth in any one of clauses <1> to <17>,

wherein the water-absorbent polymers of the absorbent layer have a fixation rate with respect to the substrate layer of preferably 10% or more, and more preferably 30% or more, and preferably 90% or less, and more preferably 80% or less.

<19> The absorbent member as set forth in any one of clauses <1> to <18>,

wherein the water-absorbent polymers of the absorbent layer have an adhesion rate of preferably 0.5 or more, and more preferably 0.7 or more, and preferably 0.95 or less, and more preferably 0.85 or less.

<20> The absorbent member as set forth in any one of clauses <1> to <19>,

wherein the content of the fibers in the absorbent layer is preferably 30 mass % or less, and more preferably 10 mass % or less relative to the total mass of the absorbent layer, and is more preferably zero, or in other words, the absorbent layer contains no fibers.

<21> The absorbent member as set forth in any one of clauses <1> to <20>,

wherein the absorbent layer is formed on each side of the substrate layer.

<22> The absorbent member as set forth in any one of clauses <1> to <21>,

wherein the content of the fibers is higher in the following order: an adjacent surface side where an adjacent surface of the entangled fiber layer that is adjacent to the absorbent layer is present; a side opposite to the adjacent surface of the entangled fiber layer; and the absorbent layer.

<23> The absorbent member as set forth in any one of clauses <1> to <22>, further comprising a cover sheet that integrally covers the absorbent sheet and the entangled fiber layer.
<24> The absorbent member as set forth in any one of clauses <1> to <23>,

wherein the substrate layer includes a plurality of strip-like substrate layers that are elongated in one direction, and

the plurality of strip-like substrate layers are disposed such that lengthwise directions thereof match, and the plurality of strip-like substrate layers are adjacent to each other in a width direction that is perpendicular to the lengthwise directions.

<25> The absorbent member as set forth in clause <24>,

wherein the plurality of strip-like substrate layers are integrated without being divided.

<26> An absorbent article comprising the absorbent member as set forth in any one of clauses <1> to <25>.
<27> The absorbent article as set forth in clause <26>,

wherein the absorbent member is disposed such that the entangled fiber layer is closer to the skin of a wearer than the absorbent sheet is.

### INDUSTRIAL APPLICABILITY

According to the present invention, it is possible to provide an absorbent member that has excellent absorbency and shape stability while being thin, and an absorbent article that includes the absorbent member.

1. An absorbent member comprising:
an absorbent sheet that includes: a substrate layer; and an absorbent layer that includes water-absorbent polymers that are fixed to one side of the substrate layer; and
an entangled fiber layer,
wherein the water-absorbent polymers of the absorbent layer and the entangled fiber layer are adjacent to each other.

2. The absorbent member according to claim 1,
wherein the water-absorbent polymers of the absorbent layer are fixed to the one side of the substrate layer using a hot melt adhesive.

3. The absorbent member according to claim 1,
wherein the substrate layer includes a non-woven fabric or a resin film.

4. The absorbent member according to claim 1,
wherein the entangled fiber layer includes pulp fibers.

5. The absorbent member according to claim 1,
wherein the entangled fiber layer includes water-absorbent polymers.

6. The absorbent member according to claim 5,
wherein the content of the water-absorbent polymers in the entangled fiber layer is 20 mass % or more and 80 mass % or less relative to the total mass of the entangled fiber layer.

7. The absorbent member according to claim 5,
wherein the content of the water-absorbent polymers in the entangled fiber layer is higher than the content of the fibers in the entangled fiber layer.

8. The absorbent member according to claim 7,
wherein the ratio between the content of the water-absorbent polymers in the entangled fiber layer and the content of the fibers in the entangled fiber layer, which is represented by the former/the latter, is 1.1 or more and 3.5 or less, on the assumption that the former>the latter.

9. The absorbent member according to claim 5,
wherein, in the entangled fiber layer, the content of the fibers is higher on an adjacent surface side where an adjacent surface that is adjacent to the absorbent layer is present than on a side opposite to the adjacent surface.

10. The absorbent member according to claim 5,
wherein, in the entangled fiber layer, the fibers are distributed concentratedly on an adjacent surface side where an adjacent surface that is adjacent to the absorbent layer is present, and the water-absorbent polymers are distributed concentratedly on a side that is opposite to the side where the absorbent layer is present.

11. The absorbent member according to claim 1,
wherein the content of the fibers in the entangled fiber layer on an adjacent surface side where an adjacent surface that is adjacent to the absorbent layer is present is 30 mass % or more and 100 mass % or less relative to the total mass of the adjacent surface side.

12. The absorbent member according to claim 1,
wherein the entangled fiber layer does not include a bonding point formed by fusion of fibers, and is a web produced using a carding method or an air-laid method, or a fiber stack of fibers defibrated from a pulp sheet.

13. The absorbent member according to claim 1,
wherein the water-absorbent polymers of the absorbent layer have a fixation rate with respect to the substrate layer of 10% or more and 90% or less.

14. The absorbent member according to claim 1,
wherein the water-absorbent polymers of the absorbent layer have an adhesion rate of 0.5 or more and 0.95 or less.

15. The absorbent member according to claim 1,
wherein the absorbent layer is formed on each side of the substrate layer.

16. The absorbent member according to claim 1,
wherein the content of the fibers is higher in the following order: an adjacent surface side where an adjacent surface of the entangled fiber layer that is adjacent to the absorbent layer is present; a side opposite to the adjacent surface of the entangled fiber layer; and the absorbent layer.

17. The absorbent member according to claim 1, further comprising a cover sheet that integrally covers the absorbent sheet and the entangled fiber layer.

18. The absorbent member according to claim 1,
wherein the substrate layer includes a plurality of strip-like substrate layers that are elongated in one direction, and
the plurality of strip-like substrate layers are disposed such that lengthwise directions thereof match, and the plurality of strip-like substrate layers are adjacent to each other in a width direction that is perpendicular to the lengthwise directions.

19. The absorbent member according to claim 18,
wherein the plurality of strip-like substrate layers are integrated without being divided.

20. An absorbent article comprising the absorbent member according to claim 1.

21-27. (canceled)
